(19) United States
(12) Patent Application Publication
(10) Pub. No.: US 2011/0095097 A1
(43) Pub. Date: Apr. 28, 2011

(54) LIQUID COMPOSITION FOR AIR FRESHENER SYSTEMS

(76) Inventors: Thomas P. Herd, Pompton Plains, NJ (US); Victor Rouchou, Chappaqua, NY (US)

(21) Appl. No.: 12/902,198

(22) Filed: Oct. 12, 2010

Related U.S. Application Data
(60) Provisional application No. 61/254,276, filed on Oct. 23, 2009.

(51) Int. Cl. A61L 9/04; A61L 9/01

(52) U.S. Cl. 239/34; 424/76.2

(57) ABSTRACT

Embodiments relate to liquid air freshener systems that allow fragrances to evaporate through use of a wicking element where fluid moves by capillary action towards the emanating surface, as well as the fragrance stabilizing compositions used therein. Cyclomethicone-based liquid compositions, preferably clear, provide an improved rate of evaporation of perfume materials from the air freshener device emanating surface and allow the use of fragrance materials with a wide range of characteristics. Embodiments utilize cyclomethicone in combination with other solvents specifically to increase solubility, enhance performance, maintain consistent fragrance character and maximize the range of aromatic materials that can be used.

### CROSS-REFERENCE TO RELATED APPLICATIONS

This application claims priority to U.S. Provisional Patent Application No. 61/254,276, filed on Oct. 23, 2009, and incorporated by reference herein.

### BACKGROUND OF THE INVENTION

1. Field of the Invention

The present subject matter relates to apparatuses and chemical mixtures for delivering fragrances within an area. More specifically, the present subject matter relates to volatile formulations used as a volatile organic compound (VOC) used in fragrances. Also discussed is a method of releasing the fragrances into the air using porous materials that wick up the fragrances via capillary action.

2. Background of the Related Art

Many different forms of fragrance delivery systems have been in use for years. Such systems include candles, heated oils, atomizers, electric and reed diffusers devices. These devices may be used in a number of applications ranging from imparting a pleasant aroma within an area, aromatherapy, and environments odor control as well as insect repellant and insecticides.

Individual known fragrance delivery systems may each have their own respective drawbacks, or overlapping or related drawbacks. Candles, for example, while effective to deliver fragrances within an environment and while often having various esthetically pleasing qualities, employ a flame which may be inappropriate in certain environments. Heated oils may also require either a flame or an electric heating element, which may pose similar drawbacks or others in particular circumstances.

Atomizer type devices may generally avoid various flame-related drawbacks associated with candles and heated oils, but such devices may require either of automated spray mechanisms (in turn requiring electrically operated pumps) or periodic manual operations that may be inconvenient or undesirable in certain environments.

Static diffuser type delivery systems avoid many of the noted drawbacks as they generally do not require heat, flame, or external energy to affect fragrance delivery, however, these too may be less effective if the diffuser liquid is not designed to provide maximum evaporation potential, to promote complete solubilization of fragrance materials and to use diluents that do not demonstrate a tendency for clogging the capillary tubes. Most diffuser devices will wick up the fragrance through capillary action. Often the fragrance liquids clog the pores of the diffuser material. The clogging of the pores may be caused by a variety of reasons, including but not limited to use of surfactants that contribute to clogging, or to chemical reactions that can cause the fragrance liquid to increase in viscosity, hindering the capillary flow of the fragrance liquid in the pores of the wicking substrate.

Creation of effective diffuser systems is further complicated by the requirement of compliance with Environmental Protection Agency (EPA) rules on atmospheric emissions. More particularly, the EPA has defined so-called “volatile organic compounds” (VOC) for purposes of preparing state implementation plans relative to attaining the national ambient air quality standards for ozone under Title I of the Clean Air Act, 42 U.S.C. §7401. VOCs include, for example, ethyl alcohol, Ethyl Acetate, Methyl Amyl Keytone, Ethyl Nonafluoroisobutyl Ether, 3-methyl-3methoxy-1-butinol, Amyl Acetate and Benzyl Formate. As an adjunct to such defined compounds, certain compounds are excluded from the definition of VOC on the basis that they make negligible contribution to any ozone formation in the troposphere. In other words, they are considered to be VOC exempt. VOC-exempt compounds include, for example linear or completely methylated siloxanes, cyclic siloxanes, branched siloxanes, methane, methylene chloride, acetone and completely fluorinated ethers.

While VOC's may have a variety of potential uses including as heat-transfer fluid or substitutes for ozone depleting substances and substances with high global warming potentials (such as hydroflurocarbons, perfluorocarbons, and perfluoropolyethers), states regulate VOC emissions as precursors to ozone formation per the above-referenced national ambient air quality standards. The desire for such regulation is based in part on the fact that tropospheric ozone (commonly known as smog) occurs when VOC and nitrogen oxides (NOX) react in the atmosphere. Because of the harmful health effects of ozone, the EPA and state governments variously limit the amount of VOC and NOX that can be released into the atmosphere. Generally speaking, the VOC's are those particular compounds of carbon (excluding certain ones) which form ozone through atmospheric photochemical reactions. Compounds of carbon (also known as organic compounds) have different levels of reactivity. In other words, they do not react at the same speed or do not form ozone to the same extent.

While various implementations of fragrance delivery systems have been developed, no design has emerged that generally encompasses all of the desired characteristics as hereafter presented in accordance with the subject technology as it relates to effective diffusion of fragrance.

### BRIEF SUMMARY OF THE INVENTION

Embodiments presented herein provide a fragrance liquid system designed to facilitate the stabilization and solubilization of fragrance materials. These fragrance materials may be, for example, crystals and terpenoids. It has been found that these and other perfumery raw materials, known to those skilled in the art, are difficult at best to solubilize. The liquid diluent system described herein has been designed to increase the evaporation rate of these and other fragrance materials in a cost effective, performance oriented formulation.

We have further discovered that the solvent blend helps stabilize some specific fragrance materials during freeze/thaw conditions. Thus, without the select solvent combination described, some fragrance materials can exhibit a tendency to drop out of solution, causing a phase-separation that does not reconstitute.

Solubilizing systems of embodiments of the invention may include, for example, dipropylene glycol dimethyl ether, 5-8% w/w, C13-14 isopariffin, 5-11% w/w, tripropylene glycol methyl ether, 2-8% w/w, bis(1-methylethyl)ester, 3-6% wt/wt and dipropylene glycol methyl ether acetate, 10-30% w/w.

For exemplary reasons, examples herein employ a reed diffuser system and wood block configuration. Those of skill in the art will recognize that other diffusion devices may be used.

In some embodiments, diffuser oil formulations have been developed using volatile organic content (VOC) exempt carriers for fragrances. The VOC-exempt carriers may be odorless, and they may be configured so that they and do not alter or obscure combined fragrances.

Air freshener diffusers including solubilizing and stabilizing compositions of the invention may provide an extended release profile for perfumes and fragrances relative to a similar delivery system that does not include the solubilizing and stabilizing compositions.

Another positive aspect of the invention is consistency of the fragrance character during evaporation over the product life. Measurements of diffuser liquid during performance testing at specific intervals, using GC/MS, shows the fragrance does not change during evaporation.

The combination of silicone, a select solvent blend and fragrance provides the synergy to maintain the fragrance character. Another present exemplary embodiment relates to an increased performance, measured by evaporation rate. Testing alongside current (2008-2009) static diffuser products, an embodiment of the invention—fragrance diffuser liquid system—exhibited a higher evaporation rate, and in some cases a linear evaporation, than all other products tested.

One skilled in the art will appreciate that modifications and variations to the specifically illustrated, referred and discussed features, elements, and steps hereof may be practiced in various embodiments and uses of the present subject matter without departing from the spirit and scope of the subject matter. Variations may include, but are not limited to, substitution of equivalent means, features, or steps for those illustrated, referenced, or discussed, and the functional, operational, or positional reversal of various parts, features, steps, or the like.

### BRIEF DESCRIPTION OF THE FIGURES

FIG. 1 demonstrates increased performance as depicted by evaporation rate provided by an embodiment of the invention as described in Example 3.

FIG. 2 shows results of weight loss comparison tests in Example 4.

FIG. 3 shows head-to-head comparison of the same fragrance oil placed in a solubilizing composition that is an embodiment of the invention vs. a typical solvent blend used in the marketplace. It shows the improved evaporation rate provided by the demonstrated embodiment, which is a 67% increased rate of evaporation.

FIG. 4 shows a Woodwick® brand diffuser.

FIG. 5 shows a typical static diffuser.

### DETAILED DESCRIPTION OF THE INVENTION

As referenced in the Summary of the Invention section, the present subject matter is particularly concerned with improved methodologies, compositions, and corresponding apparatuses for delivering fragrances to environments by diffusion.

There are several particular aspects to fragrance delivery systems as herein described in accordance with the present technology that contribute to commercial as well as functional success for such corresponding products. For example, more generally speaking, it is significant for users that any volatile components used be VOC-exempt or used at levels within the guidelies relative to EPA requirements and regulations, as referenced above. Further, it is desirable that such fluids themselves be odorless so as not to obscure or add to any combined fragrance. Additionally, room temperature evaporation rates should preferably be such that any corresponding or resulting product will be effective for an extended period of time while maintaining sufficient fragrance transport into a surrounding environment. It is generally preferred that such fragrance transport formulations should be effective for relative duration of time, such as, for example, one to two months.

Applicants have found that cyclomethicone fluids, when presented and used per the present technology, demonstrate properties that contribute toward such aspects. One example of a particular such cyclomethicone fluid is produced by Dow Corning® and commercially available as Dow Corning® 245 Fluid. DC-245 Fluid is a volatile cyclomethicone fluid that is clear, odorless, VOC exempt, has low toxicity and low surface tension fluid, and evaporates completely at room temperature. Other cyclomethicone fluids suitable for use with the present technology include, but are not limited to: Dow Corning® 344 Fluid, Dow Corning® 246 Fluid, Dow Corning® 345 Fluid, and Dow Corning® 200 Fluid.

While those of ordinary skill in the art will appreciate the general concepts of reed diffusers, our present technology provides significantly improved fragrance delivery achieved through exemplary formulations of diffuser oil composition. More specifically, our present technology may make advantageous use of volatile cyclomethicone fluids, blended with other fluids as described herein, as a fragrance transport mechanism. Such fluids are particularly useful because they are odorless, volatile organic content (VOC) exempt, low in toxicity, low in surface tension, and highly effective for complete evaporation at room temperature. As a result, a fragrance can be dissolved into such fluids resulting in a desired product having similar advantageous properties.

An exemplary formulation in accordance with our present technology may include fragrance, 10-18% w/w; cyclomethicone fluid, 50-60% w/w; dipropylene glycol methyl ether acetate, 18-23% w/w, C13-14 isopariffin, 7-11% w/w, dimethyl siloxane hydroxyalkyl-terminated, 2-4% w/w and 3-methoxy-3-methyl-1-butanol, 8-13% w/w.

Optional materials that may be include in the formulation are propylene glycol n-butyl ether, 5-14% w/w, 2-(2-butoxy-ethoxy)ethanol, bis(1-methylethyl)ester, 3-6% wt/wt, 2-7% w/w and dipropylene glycol n-butyl ether, 1-5% w/w. Those skilled in the art will recognize that additional materials may be added as desired.

The select solvents, cyclomethicone and fragrance blend is employed not only to address costing aspects but also to address desired solubility, stability and performance aspects of the invention. Suitable solvent/carriers may be selected from materials including, but not limited to, cyclopentasiloxane, C13-14 isoparaffin, 3-methoxy-3-methyl-1-butanol, cyclotetrasiloxane, tripropylene glycol monomethyl ether, bis(1-methylethyl)ester, cyclomethicone, dipropylene glycol methyl ether acetate or a mixture thereof.

Stability tests were conducted throughout the development period where current market reed diffuser products were used for comparison against the invention. The market diffuser products exhibited stability issues with spice, citrus and vanilla fragrance compositions. Initially, the invention exhibited similar, but reduced instability. Further development and combination of C13-14 isopariffin, 28-32% wt/wt and dipropylene glycol methyl ether acetate, 67-75% wt/wt, resolved these stability problems.

Embodiments of the invention may provide diffusers with enhanced fragrance release profiles. For example, the increased stabilization and solubilization of the fragrance may allow a fragrance release having a linear profile that is constant or relatively constant over time.

A variety of diffusing devices may be used. For example, a diffuser comprised of rattan reeds of 6-10″ length, a glass container with an opening at the top approximately 1″ in diameter and solvent or solvent blend may be used. Static or passive diffuser systems in basic format must have a container with an opening at the top, a solvent or solvent blend and some device for transport (rattan reed, wood dowel, membrane) of the solvent/solvent blend to the emanating surface.

Diffusers constructed from wood blocks or different types of wood laminated together are also suitable. In one embodiment the diffuser construction may be comprised of a container, dowel and wooden lid.

Various fragrances may be used. They include, for example, but are not limited to Vanilla Bean, Cinnamon Chai, Frasier Fir, Linen, Spice Clove, Fireside, Redwood, Pumpkin Butter, Applewood and Citrus & Herbs.

### EXAMPLES

### Example 1

### Preparation of a Solubilizing Composition

This example reports the preparation of a solubalizing composition that are in embodiments of the invention. The embodiment prepared in this example was then used in the testing presented in the further examples in this application.

The solubilizing system may be prepared without any external heat required. The process begins with a fragrance added to C13-14 isoparaffin and allowed to mix thoroughly. This mixture is blended with dipropylene glycol methyl ether acetate and dimethyl siloxane hydroxyalkyl-terminated. Another example of a solubilizing composition of similar effect is achieved by combining fragrance, tripropylene glycol methyl ether and 3-methoxy-3-methyl-1-butanol.

### Example 2

### Testing of a Solubilizing Composition of the Invention with Vanilla Fragrance

Typically, fragrances having high crystal, spice, citrus and terpene levels have, in the past, presented solubility and stability issues in the past in typical static diffuser liquids. Embodiments of the invention may resolve these issues to allow higher levels of these materials be used in the fragrance oil.

Vanilla type fragrances typically incorporate high levels of crystals that present solubility issues in evaporative systems. Therefore, vanilla fragrances seemed to be an ideal candidate to test the efficacy of embodiments of the invention.

Full stability testing was conducted to insure no permanent adverse effects were observed.

Test parameters included exposure in the following stability chambers; 25° C., 37° C., 45° C., 50° C., 5° C., UV Light, CWL Light and 3 Cycles freeze/thaw [−20° C./25° C.].

An example of a vanilla fragrance with high crystal content that has successfully been incorporated; ingredients parts by weight: 1) Aldehyde C-18 0.4%, 2) Anisic Aldehyde 4%, 3) Coumarin 10%, 4 Ethyl Maltol 4% 5) Ethyl Vanillin 18%, 6) Oxyphenylon 0.5%, 7) Vanillin USP 17%, 8) Benzyl Benzoate 46.1%.

### Example 3

### Solvent Blend Comparative Testing of the Invention to Demonstrate Superior Performance

A test was designed to compare existing market product solvent blend with the invention to demonstrate the superior performance achieved by embodiments of the invention. Evaporation rate trials were selected as the procedure for this evaluation. Test parameters and conditions: GC/MS analysis of static diffuser market products were performed to determine the solvent or solvent blends used in their product. Samples were prepared using a Linen fragrance oil (0066355A) at 15% w/w in the solvent or solvent blends representing 85% w/w. Five (5) test samples of each at 65 grams w/w were prepared in identical glass containers using 10 each of the same 3.0 mm diameter rattan reeds of 8″ length. Initial weights taken and samples weighed at 1 week intervals, evaporation test room conditions; 72° F., 63% RH.

Analysis of static diffuser market products used for comparative testing showed the following compositions: Pier 1 products use Carbitol® blended with the fragrance; Febreze uses dipropylene glycol methyl ether acetate, 80% w/w with tripropylene glycol methyl ether 20% w/w and this mixture is blended with fragrance at a ratio of 85:15 wt/wt.; Glade brand solvent blend is 3-methoxy-3-methyl-1-butanol, 12% wt/wt with dipropylene glycol methyl ether acetate, 88% wt/wt and blended with fragrance at a ratio of 88:12.

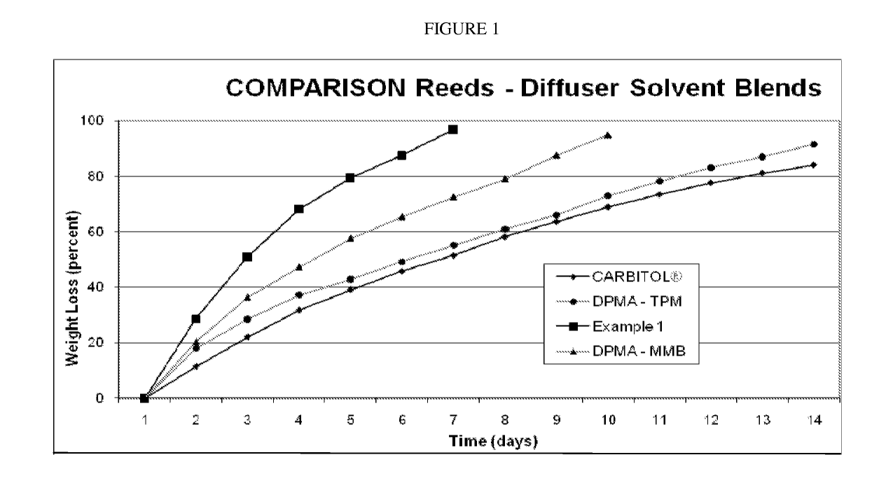

FIG. 1 demonstrates increased performance in head-to-head comparison as depicted by evaporation rate provided by an embodiment of the invention as described by the testing above.

### Example 4

### Testing of a Solubilizing Composition of the Invention with Spice Fragrance

Spice type fragrances exhibit different issues of stability in freeze/thaw conditions. Testing was conducted to evaluate and insure stability in freeze/thaw conditions; 3 cycles, 24 hours @ −20° C. then 24 hours @ 24° C. No permanent adverse effects were observed.

An example of a Spice Clove fragrance with high levels of spice related materials that exhibits stability and compatibility issues in silicone and silicone blends, but has successfully been incorporated into the invention without exhibiting any of the stated stability issues; fragrance ingredients parts by weight: 1) Acetophenone 0.2%, 2) Acetyl Pyrazine 0.1%, 3) Cyclotene 0.7%, 4) Cinnamic Alcohol 5.5%, 5) Indolal 0.3%, 6) Amyl Phenyl Acetate-Iso 0.3%, 7) Nutmeg Oil East Indian 0.3%, 8) Tetrahydro Linalool 0.2%, 9) Terpinyl Acetate 4.0%, 10) Phenyl Acetaldehyde @ 50% PEA 0.2%, 11) Coumarin 2.2%, 12) Eugenol USP 12.0%, 13) Hedione 0.2%, 14) Hexyl Cinnamic Aldehyde 0.2%, 15) Methyl Cinnamic Aldehyde 6.5%, 16) Dermol DOA 35.3%, 17) Cinnamic Aldehyde 11.5%, 18) Benzyl Benzoate 20.0%, 19) Methyl Anthranilate 0.3%.

### Example 5

### Testing of Fragrance Character Consistency of the Invention over Functional Life

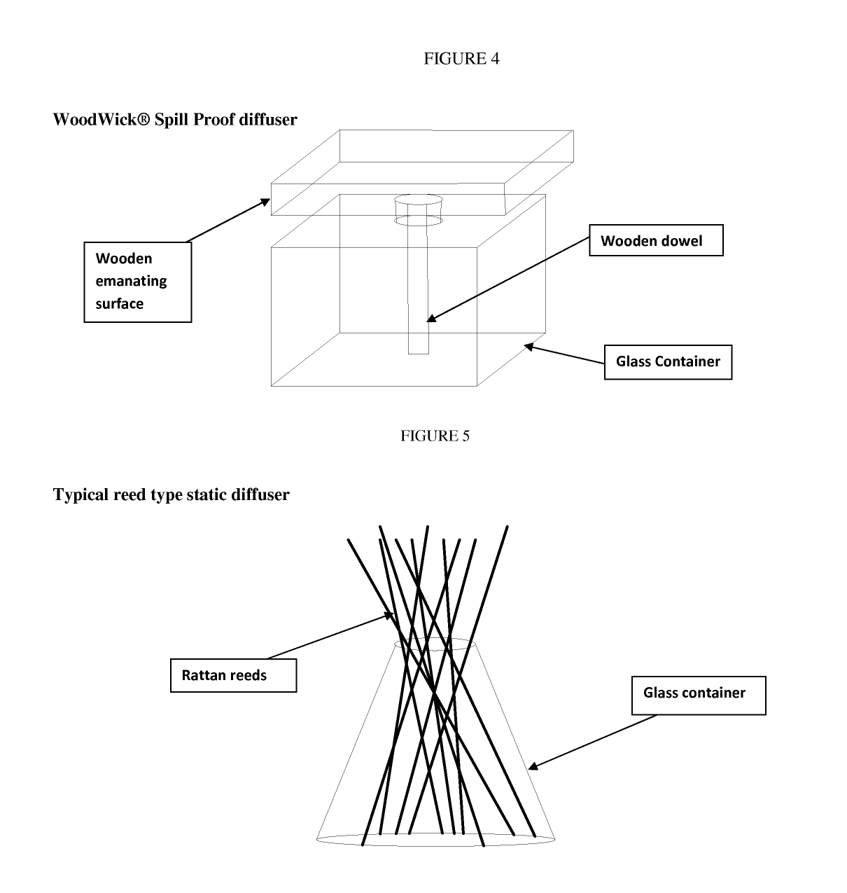

Testing was conducted to confirm fragrance character consistency does not during the functional life of static diffuser products using and embodiment the invention. WoodWick® Spill Proof diffuser samples were prepared using an embodiment of the invention with fragrance level at 15% w/w. The WoodWick® Spill Proof diffuser consists of a cube-like glass container (FIG. 4) to hold the diffusing solution, a dowel for transport of the liquid to the emanating surface and a wood block as the emanating surface. Evaporation trials were conducted and at specific intervals, small samples of the product inside the container were extracted and submitted for analysis the using a Gas Chromatograph (GC). Analytical results demonstrated that the fragrance character did not change over the life of the product.

The charts below are the results of an analysis made by Gas Chromatograph of three (3) distinctly different fragrance compounds; a Linen Type, Herbal Type and Fruity Type, that were incorporated at 15% w/w into an embodiment of the invention.

| | | | | | |
|---|---|---|---|---|---|
| | Peak % | Peak % | Peak % | Peak % | Standard |
| RI Value | (Day 0) | (Day 7) | (Day 21) | (Day 28) | Deviation |

Linen 0066355A (Linen Type)

| | | | | | |
|---|---|---|---|---|---|
| 517.8 | 12.78 | 13.13 | 11.84 | 12.93 | 0.57 |
| 639.4 | 1.50 | 1.51 | 1.46 | 1.55 | 0.04 |
| 676.2 | 1.52 | 1.54 | 1.47 | 1.53 | 0.03 |
| 704.9 | 2.01 | 2.04 | 2.07 | 1.98 | 0.04 |
| 707.4 | 1.01 | 1.05 | 0.75 | 1.02 | 0.14 |
| 757-791 | 66.67 | 66.50 | 67.18 | 66.66 | 0.29 |
| 839.8 | 1.17 | 1.20 | 1.18 | 1.13 | 0.03 |
| 892.7 | 0.94 | 0.95 | 0.91 | 0.96 | 0.02 |
| 937.8 | 0.79 | 0.76 | 0.77 | 0.78 | 0.01 |
| 960.6 | 0.35 | 0.34 | 0.37 | 0.35 | 0.01 |
| 974.4 | 1.53 | 1.55 | 1.67 | 1.56 | 0.06 |
| 1021.3 | 0.84 | 0.85 | 0.82 | 0.87 | 0.02 |
| 1053.3 | 0.68 | 0.69 | 0.67 | 0.71 | 0.02 |
| 1088.2 | 0.92 | 0.93 | 0.90 | 0.95 | 0.02 |
| 1197.1 | 0.43 | 0.43 | 0.42 | 0.44 | 0.01 |
| 1239.6 | 1.22 | 1.21 | 1.21 | 1.29 | 0.04 |
| 1352 | 2.03 | 2.00 | 2.03 | 2.21 | 0.10 |
| 1379.9 | 0.63 | 0.62 | 0.63 | 0.69 | 0.03 |
| 1607.6 | 0.61 | 0.59 | 0.62 | 0.69 | 0.04 |

Meadow 01271159B (Herbal Type)

| | | | | | |
|---|---|---|---|---|---|
| 518.6 | 13.43 | 12.68 | 12.53 | 12.97 | 0.39 |
| 702-708 | 6.55 | 6.26 | 5.96 | 6.20 | 0.24 |
| 753-798 | 67.89 | 68.51 | 69.31 | 68.74 | 0.59 |
| 831.5 | 0.58 | 0.57 | 0.60 | 0.54 | 0.02 |
| 833.8 | 0.30 | 0.29 | 0.34 | 0.29 | 0.03 |
| 895 | 0.38 | 0.37 | 0.30 | 0.31 | 0.04 |
| 952.2 | 0.99 | 0.92 | 0.88 | 0.94 | 0.05 |
| 1021.9 | 2.09 | 1.98 | 1.90 | 2.04 | 0.08 |
| 1119.1 | 0.29 | 0.28 | 0.26 | 0.29 | 0.01 |
| 1171.8 | 0.31 | 0.31 | 0.29 | 0.31 | 0.01 |
| 1239.9 | 1.30 | 1.26 | 1.23 | 1.31 | 0.04 |
| 1352.3 | 2.25 | 2.24 | 2.06 | 2.31 | 0.11 |

Currant 00411147B (Fruity Type)

| | | | | | |
|---|---|---|---|---|---|
| 517.7 | 12.92 | 12.38 | 13.15 | 12.06 | 0.50 |
| 639.6 | 5.05 | 4.93 | 4.97 | 4.79 | 0.11 |
| 678.4 | 0.47 | 0.47 | 0.47 | 0.46 | 0.01 |
| 703.4 | 1.23 | 1.21 | 1.28 | 1.18 | 0.04 |
| 763-800 | 61.72 | 61.99 | 61.37 | 62.41 | 0.44 |
| 807.5 | 2.07 | 2.07 | 2.13 | 1.98 | 0.06 |
| 836.7 | 0.40 | 0.45 | 0.42 | 0.37 | 0.03 |
| 855.4 | 0.29 | 0.37 | 0.29 | 0.36 | 0.04 |
| 859 | 0.42 | 0.45 | 0.45 | 0.51 | 0.04 |
| 863.3 | 0.32 | 0.31 | 0.34 | 0.32 | 0.01 |
| 898.8 | 0.23 | 0.28 | 0.22 | 0.28 | 0.03 |
| 950.8 | 0.31 | 0.29 | 0.34 | 0.29 | 0.02 |
| 1024.4 | 1.04 | 1.00 | 1.09 | 0.98 | 0.05 |
| 1088.5 | 2.92 | 2.84 | 3.09 | 2.75 | 0.14 |
| 1.618 | 1.88 | 1.85 | 1.81 | 1.62 | 0.12 |
| 1133.3 | 0.37 | 0.39 | 0.39 | 0.41 | 0.01 |
| 1242.5 | 4.50 | 4.42 | 4.66 | 4.31 | 0.15 |
| 1456.4 | 0.10 | 0.10 | 0.11 | 0.94 | 0.42 |

In the charts above, RI Value is the relative index value of specific raw material in the fragrance compound as identified by Gas Chromatograph. Peak % represents the measured area under each of the peaks on the GC chart from analysis of this product. Standard Deviation relates to the average change in concentration of the material in the product over the duration of the test. These results document that the fragrance character does not change over the life of the product.

### Example 6

### Performance evaluations demonstrating superiority of the Invention over Functional Life

Comparative evaporation studies were conducted against actual market reed diffuser products to evaluate evaporation potential and demonstrate the superior performance achieved by the invention. Market products selected were in 3 fragrance categories; Linen Type, Herbal Type and Fruity Type. Evaporation rate trials were selected as the procedure for this evaluation. Five (5) test samples of each product were purchased from various stores; Linen Type—Yankee Candle Brand Clean Cotton, Febreze® Willow Blossom, Glade Brand Sheer White Cotton and WoodWick® Linen; Herbal Type—Yankee Candle Sage & Citrus, Febreze® Green Tea Citrus, Glade Lotus Bamboo and WoodWick® Meadow; Fruit Types—Yankee Candle Macintosh, Febreze® Pomegranate Mango, Glade Currants & Acai and WoodWick® Currant were placed in a room measuring 10′ by 10′ with temperature set at 72° F. and RH 63%. Samples were evaluated for weight loss weekly.

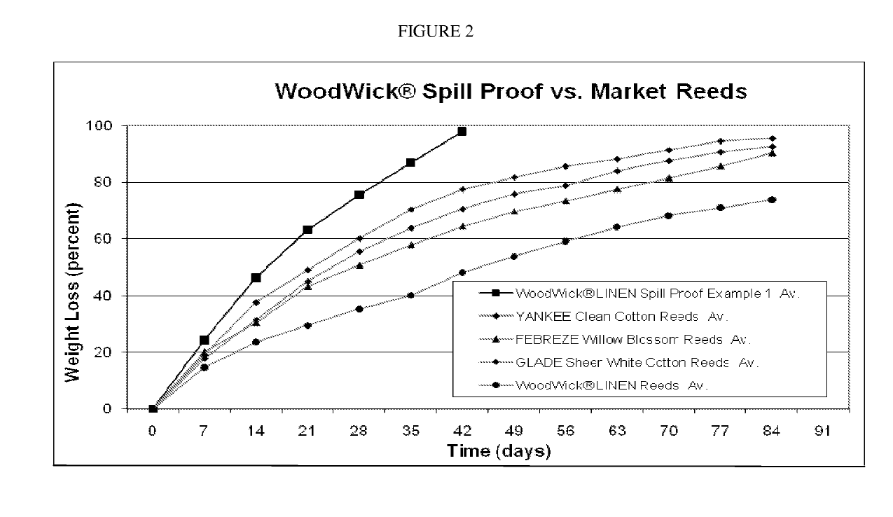

The WoodWick® reed diffuser uses the invention and was compared to the leading market reed diffuser samples listed above. FIG. 2 shows the results of these weight loss comparison trials.

### Example 7

### Head-to-Head comparative evaluations demonstrating superiority of the Invention over Functional Life

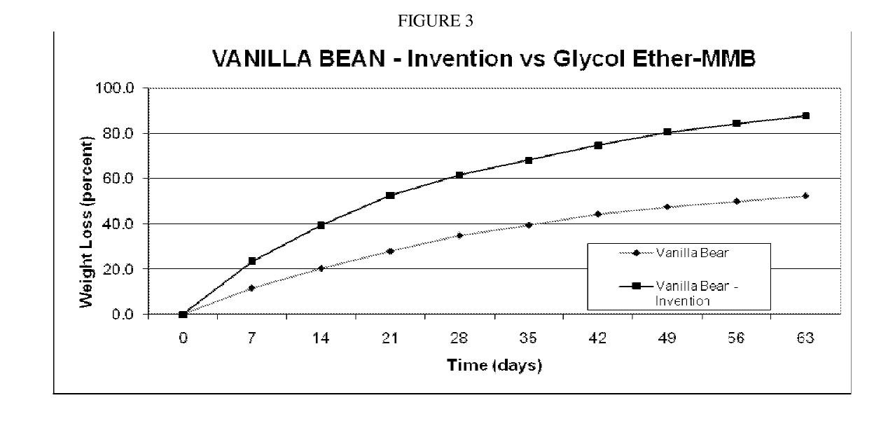

A head-to-head comparative evaporation study was conducted using a Vanilla Bean fragrance oil (0057641) at 15% wt/wt in the invention and at 15% wt/wt in a Glycol Ether/MMB blend consisting of 75% wt/wt Glycol Ether and 25% wt/wt MMB. FIG. 3 shows the results after 63 days in test where the invention provided 67% a greater evaporation rate.

I claim:

1. An air freshener diffuser comprising:
a vessel holding a diffusing composition, said diffusing composition comprising
8.0% to 20% w/w of a perfume having a fragrance character;
a stabilizing composition comprising cyclopentasiloxane, dipropylene glycol methyl ether acetate, C13-14 isopariffin, 3-methoxy-3-methyl-1-butanol, dimethyl siloxane hydroxyalkyl-terminated, cyclotetrasiloxane, and cyclomethicone; and
an emanating surface providing diffusion of said diffusing composition.

2. The air freshener diffuser of claim 1, further comprising at least one wicking element with a cellular structure extending the length of said wicking element.

3. The air freshener diffuser of claim 1, wherein said stabilizing composition increases the solubility potential of said diffusing composition relative to the solubility potential of the diffusing composition without the stabilizing composition.

4. The air freshener diffuser of claim 1, wherein said diffusing composition has a flash point equal to or greater than 104° F. but not more than 200° F.

5. The air freshener diffuser of claim 1, wherein said compound is cyclomethicone.

6. The air freshener diffuser of claim 1, wherein said stabilizing composition is odorless and does not alter or obscure the fragrance character.

7. The air freshener diffuser of claim 1, wherein said diffuser delivers said fragrance at a level of 40-60% greater than that of an otherwise identical air freshener diffuser without said stabilizing composition over a time period of between 45-60 days.

8. A diffusing fragranced composition comprising 12-18% fragrance, 10-18% a volatile organic compound (VOC), and 57-71% solvent carrier, wherein said VOC has a reactivity with nitrogen oxides less than or equal to that of ethane.

9. A fragrance solubilizing composition comprising cyclomethicone and one or more compounds selected from the group consisting of cyclopentasiloxane, C13-14 isoparaffin, 3-methoxy-3-methyl-1-butanol, cyclotetrasiloxane, tripropylene glycol monomethyl ether, dipropylene glycol methyl ether acetate or a mixture thereof.

10. The air freshener diffuser of claim 1, wherein said emanating surface is a wicking element selected from the group consisting of at least one reed, each reed having a cellular structure with a diameter of 2.8-4.5 mm extending the length of the wicking element, and at least one wood block, each wood block having a square or rectangular cross-section, a thickness between 1 and 2 inches, and a surface area of 5-9 square inches.

11. A fragrance solubilizing composition consisting essentially of cyclopentasiloxane, dipropylene glycol methyl ether acetate, C13-14 isopariffin, 3-methoxy-3-methyl-1-butanol, dimethyl siloxane hydroxyalkyl-terminated, cyclotetrasiloxane, and cyclomethicone.

12. The fragrance solubilizing composition of claim 9, comprising cyclopentasiloxane, cyclotetrasiloxane, cyclomethicone fluid, 50-60% w/w; dipropylene glycol methyl ether acetate, 18-23% w/w, C13-14 isopariffin,75-11% w/w, dimethyl siloxane hydroxyalkyl-terminated, 2-4% w/w and 3-methoxy-3-methyl-1-butanol, 8-13% w/w.

13. The fragrance solubilizing composition of claim 9, further comprising at least one compound selected from the group consisting of propylene glycol n-butyl ether, 2-(2-butoxy-ethoxy)ethanol, and dipropylene glycol n-butyl ether.

14. The air freshener diffuser of claim 1, wherein said perfume is a vanilla fragrance comprising aldehyde C-18, anisic aldehyde, coumarin, ethyl maltol, ethyl vanillin, oxyphenylon, Vanillin USP, and benzyl benzoate.

15. The air freshener diffuser of claim 1, wherein said perfume is a spice clove fragrance comprising acetophenone, acetyl pyrazine, cyclotene, cinnamic alcohol, indolal, amyl phenyl acetate-iso, nutmeg oil East Indian, tetrahydro linalool, terpinyl acetate, phenyl acetaldehyde @ 50% PEA, coumarin, eugenol USP, hedione, hexyl cinnamic aldehyde, methyl cinnamic aldehyde, dermol DOA, cinnamic aldehyde, benzyl benzoate, and methyl anthranilate.
